(19) United States
(12) Patent Application Publication
(10) Pub. No.: US 2024/0363198 A1
(43) Pub. Date: Oct. 31, 2024

(54) SYSTEM FOR IDENTIFYING GENETIC VARIANTS AND METHOD THEREOF

(71) Applicant: CARDIAI TECHNOLOGIES, Calgary (CA)

(72) Inventors: Minal Borkar TRIPATHI, Calgary (CA); Sidharth Singh BHINDER, Calgary (CA); Anmol Singh KAPOOR, Calgary (CA); Amrita KAUR, Calgary (CA)

(21) Appl. No.: 18/646,889

(22) Filed: Apr. 26, 2024

Related U.S. Application Data
(60) Provisional application No. 63/498,601, filed on Apr. 27, 2023.

(51) Int. Cl. G16B 30/00; G16B 45/00; G16H 50/30

(52) U.S. Cl. CPC G16B 30/00; G16B 45/00; G16H 50/30

(57) ABSTRACT

The present disclosure relates to a computer-implemented method for identifying genetic variants associated with a complex trait or disease includes obtaining genetic data from a population, storing the genetic data obtained from a population, processing the genetic data to identify genetic variant, identifying genetic variants, including single nucleotide polymorphisms (SNPs), insertions, deletions, and structural variations. The method also includes using statistical analysis to identify genetic variants that are associated with the trait or disease of interest and visualizing the results through an interactive graphical interface.

### CROSS-REFERENCE TO RELATED APPLICATIONS

This application claims the benefit of U.S. Provisional Application No. 63/498,601 titled “SYSTEM FOR IDENTIFYING GENETIC VARIANTS” filed by the applicant on Apr. 27, 2023, which is incorporated herein by reference in its entirety.

### FIELD OF THE INVENTION

Embodiments of the present invention relate to the field of genomics and more particularly, relate to a method and system for identifying genetic variants associated with complex traits and diseases.

### DESCRIPTION OF THE RELATED ART

As human genome contains millions of genetic variations, including single nucleotide polymorphisms (SNPs), insertions/deletions (indels), and structural variants. These variations contribute to the diversity observed in traits and diseases among individuals. Many personal traits and diseases, such as height, body mass index (BMI), diabetes, cardiovascular diseases, and psychiatric disorders, are influenced by multiple genetic and environmental factors. Understanding the genetic basis of these complex traits requires large-scale studies that examine genetic variations across the entire genome.

Genome-wide association studies (GWAS) have revolutionized the field of genetics by enabling researchers to investigate the genetic basis of complex traits and diseases. The main aim of GWAS is to identify genetic variations associated with specific traits, diseases, or phenotypes across the entire genome. The development of high-throughput genotyping and sequencing technologies has made it feasible to genotype hundreds of thousands to millions of genetic variants in large cohorts of individuals cost-effectively. This has paved the way for conducting GWAS on a genome-wide scale.

GWAS may help researchers identify genetic variants that are statistically associated with a trait or disease. By pinpointing genetic variants associated with diseases, GWAS provide insights into the molecular mechanisms and pathways involved in disease development. This knowledge is crucial for developing targeted therapies and interventions. In addition, GWAS may contribute to the field of personalized medicine by identifying genetic markers that can predict individual disease risk, treatment response, and drug efficacy. This allows for more tailored and effective healthcare strategies.

Furthermore, GWAS shed light on population-specific genetic variations and differences in disease prevalence. They help in understanding genetic diversity among populations and contribute to studies on evolutionary genetics and population health. Findings from GWAS can inform drug discovery efforts by highlighting potential drug targets based on genetic associations with disease pathways.

However, the interpretation of GWAS results is not always straightforward. GWAS involve comparing the genomes of individuals with a particular trait or disease to those without the trait or disease, searching for genetic variations that are more common in one group than the other. While GWAS have identified many genetic variants associated with complex traits and diseases, the challenge lies in determining which variants are actually causal and which are merely associated.

GWAS typically identify a large number of genetic variants associated with the trait or disease of interest, often numbering in the hundreds or thousands. To identify the causal variants among these associations, researchers must use a variety of statistical and computational techniques to narrow down the list of potential candidates. This can involve examining the functional effects of the variants, assessing their frequency in different populations, and investigating their association with other traits or diseases.

Moreover, the interpretation of GWAS results requires specialized knowledge and expertise. Researchers must be able to understand the underlying biology of the trait or disease being studied, as well as the complex interactions between genetic and environmental factors. In addition, they must be able to navigate complex statistical and computational methods for analyzing large amounts of genomic data.

Despite these challenges, the identification of genetic variants associated with complex traits and diseases has enormous potential for advancing our understanding of human biology and improving healthcare. By identifying the underlying genetic causes of diseases, we can develop more targeted and effective treatments and improve patient outcomes.

Accordingly, to overcome the disadvantages of the prior art, there is an urgent need for a technical solution that overcomes the above-stated limitations in the prior arts. The present invention provides a method and system for identifying genetic variants associated with complex traits and diseases.

### SUMMARY OF THE INVENTION

The present disclosure solves all the above major limitations of a method and system for identifying genetic variants associated with complex traits and diseases. Further, the present disclosure ensures that the disclosed invention may fulfil following aspects:

An aspect of the present disclosure is to provide an effective and reliable method for identifying genetic variants associated with complex personality traits and diseases.

Another aspect of the present disclosure is to provide a less complex method for identifying genetic variants.

Another aspect of the present disclosure is to provide a cost-effective method for identifying genetic variants.

Another aspect of the present disclosure is to provide a resource-efficient method for identifying genetic variants.

Another aspect of the present disclosure is to provide a method for identifying genetic variants that has high sensitivity and specificity.

Another aspect of the present disclosure is to provide a method for identifying genetic variants that can aid in examining the gene's allelic diversity and variations specific to a particular population.

Another objective of the present disclosure is to provide a method for identifying a particular gene that can cover a wide range of genetic variations.

Another aspect of the present disclosure is to provide a method for identifying a particular gene that has enhanced utility and applicability.

Another aspect of the present disclosure is to provide a method for identifying a particular gene that can handle large-scale genetic data efficiently.

Another aspect of the present disclosure is to provide a system for identifying a particular gene that is scalable.

Another aspect of the present disclosure is to provide a system for identifying a particular gene that can minimize processing time and resource requirements while maintaining accuracy and reliability.

Another aspect of the present disclosure is to provide a system that can provide information about variant type, genomic location, functional impact, allele frequencies, and potential disease associations to facilitate downstream analysis and interpretation.

Yet another aspect of the present disclosure is to provide a system for identifying a particular gene that can effectively handle genetic data from diverse populations.

Yet another aspect of the present disclosure is to provide a system for identifying a particular gene that can find application in genetic research, clinical applications, and personalized medicine initiatives.

Yet another aspect of the present disclosure is to provide a method for identifying a particular gene that has a user-friendly interface that allows researchers, clinicians, or end-users to easily utilize the method, input data, adjust parameters, visualize results, and interpret findings.

Embodiments of the present invention relate to a computer-implemented method for identifying genetic variants associated with a complex trait or disease. The method includes obtaining genetic data from a population. The method also includes storing the genetic data obtained from a population. The method also includes processing the genetic data to identify genetic variants. The method also includes identifying genetic variants, including single nucleotide polymorphisms (SNPs), insertions, deletions, and structural variations. The method also includes using statistical analysis to identify genetic variants that are associated with the trait or disease of interest. The method also includes visualizing the results through an interactive graphical interface.

In accordance with an embodiment of the present invention, the genetic data is obtained using a technique comprising next-generation sequencing, microarray analysis, polymerase chain reaction (PCR) and so forth.

In accordance with an embodiment of the present invention, the processing of the stored genetic data includes aligning the genetic data to a reference genome to identify sequence variations.

In accordance with an embodiment of the present invention, the processing of the stored genetic data includes annotating the identified variants with genomic features such as gene annotations, functional domains, and regulatory elements.

In accordance with an embodiment of the present invention, the visualization of the results includes generating visual representations of variant data, including frequency plots, genotype-phenotype correlations, and pathway enrichment maps.

Another embodiment of the present invention, a computer-implemented system for identifying genetic variants associated with a complex trait or disease. The system comprising a data collection module. The system also comprising a data storage module linked to the data collection module and the data storage module configured to store genetic data obtained from a population. The system also comprising a processing assembly linked to the data storage module and the processing assembly further comprises a variant identification module linked to the data storage module and the variant identification module is configured to process the genetic data to identify genetic variants in the population. The processing assembly also comprising a statistical analysis module linked to the variant identification module and the statistical analysis module configured to analyze the genetic variants identified by the variant identification module and determine which variants are associated with the trait or disease of interest. The processing assembly also comprising a visualization module linked to the statistical analysis module and the visualization module configured to generate visualizations of the genetic variants identified by the variant identification module and the statistical analysis results generated by the statistical analysis module. The system also comprising a graphical interface linked to the visualization module and the graphical interface configured to visualizing the results obtained. The system also comprising a communication network linking the data collection module to the data storage module, the communication network configured to enable the transfer of raw genetic data and facilitate integration with external computing resources.

In accordance with an embodiment of the present invention, the data collection module collects genetic data including genomic data, transcriptomic data, epigenomic data, proteomic data, and/or any combination thereof.

In accordance with an embodiment of the present invention, the genetic data variants include single nucleotide polymorphisms (SNPs), copy number variations (CNVs), insertions, deletions, or any other type of genetic variation.

In accordance with an embodiment of the present invention, the data storage module for the genetic data is configured to be scalable and reliable storage solution capable of handling large volumes of genetic data.

In accordance with an embodiment of the present invention, the data storage module for the genetic data is configured to be efficient storage enabling retrieval of large volumes of genetic data while maintaining data integrity.

In accordance with an embodiment of the present invention, the variant identification module uses suitable technique to identify genetic variants, such as, read alignment, variant calling, and/or haplotype phasing.

In accordance with an embodiment of the present invention, the statistical analysis module uses any suitable technique to perform statistical analysis, including logistic regression, linear regression, or bayesian analysis.

In accordance with an embodiment of the present invention, the statistical analysis module is configured to correct for population stratification.

In accordance with an embodiment of the present invention, the statistical analysis module is configured to perform genome-wide multiple testing correction.

In accordance with an embodiment of the present invention, the statistical analysis module is configured to incorporate functional annotation information.

In accordance with an embodiment of the present invention, the visualization module facilitates interpretation of the statistical analysis result and identification of potential functional mechanisms underlying the genetic variants.

### BRIEF DESCRIPTION OF THE DRAWINGS

So that the manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

The invention herein will be better understood from the following description with reference to the drawings, in which:

FIG. 1 is a flowchart illustrating a computer-implemented method for identifying genetic variants associated with a complex trait or disease, in accordance with an embodiment of the present invention; and

FIG. 2 is a block diagram illustrating a computer-implemented system for identifying genetic variants associated with a complex trait or disease, in accordance with an embodiment of the present invention.

The method for identifying genetic variants and system thereof is illustrated in the accompanying drawings, which like reference letters indicate corresponding parts in the various figures. It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE INVENTION

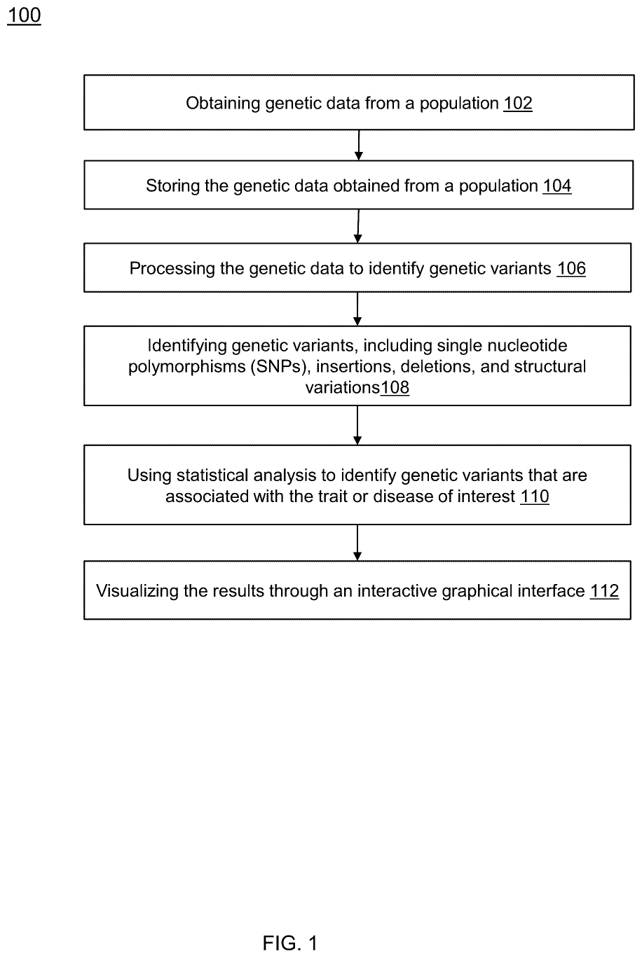

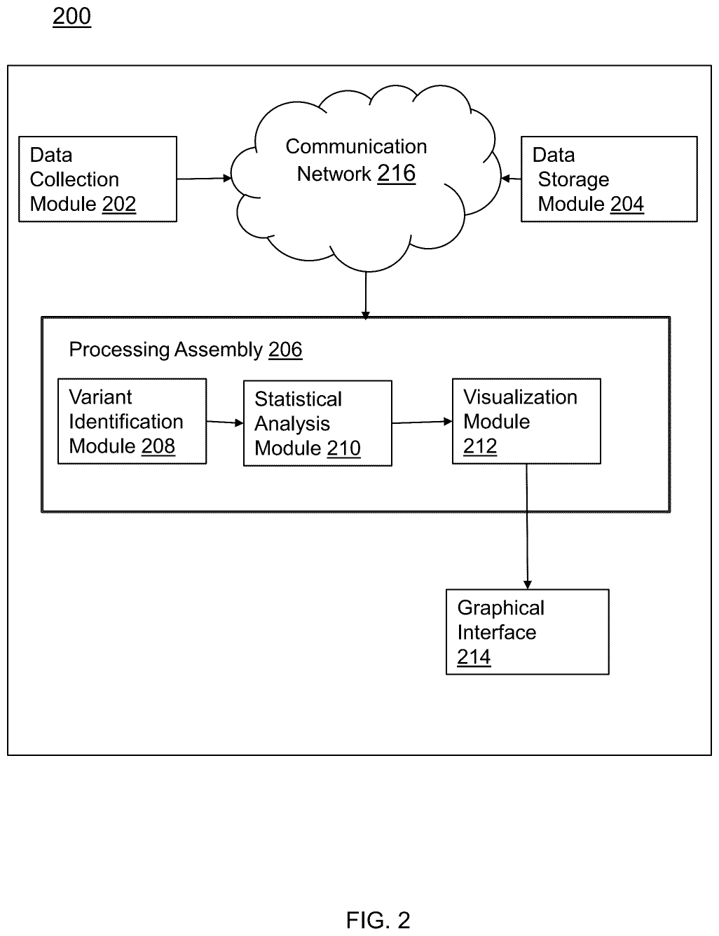

The principles of the present invention and their advantages are best understood by referring to FIGS. 1 and 2. In the following detailed description, numerous specific details are set forth in order to provide a thorough understanding of the embodiment of the invention as illustrative or exemplary embodiments of the invention, specific embodiments in which the invention may be practiced are described in sufficient detail to enable those skilled in the art to practice the disclosed embodiments. However, it will be obvious to a person skilled in the art that the embodiments of the invention may be practiced with or without these specific details. In other instances, well-known methods, procedures and components have not been described in detail so as not to unnecessarily obscure aspects of the embodiments of the invention.

The following detailed description is, therefore, not to be taken in a limiting sense, and the scope of the present invention is defined by the appended claims and equivalents thereof. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. References within the specification to “one embodiment,” “an embodiment,” “embodiments,” or “one or more embodiments” are intended to indicate that a particular feature, structure, or characteristic described in connection with the embodiment is included in at least one embodiment of the present invention.

Although the terms first, second, etc. may be used herein to describe various elements, these elements should not be limited by these terms. These terms are generally only used to distinguish one element from another and do not denote any order, ranking, quantity, or importance, but rather are used to distinguish one element from another. Further, the terms “a” and “an” herein do not denote a limitation of quantity, but rather denote the presence of at least one of the referenced items.

The conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

FIG. 1 is a flowchart illustrating a computer-implemented method 100 for identifying genetic variants associated with a complex trait or disease, in accordance with an embodiment of the present invention. The method 100 may comprise following steps.

At 102, obtaining genetic data from a population. The genetic data may be obtained using any suitable technique comprising limited to next-generation sequencing, microarray analysis, or polymerase chain reaction (PCR).

In some embodiments, sample collection is performed to obtain genetic data. The sample collection may be performed using random sampling, stratified sampling, or targeted sampling and/or combination thereof. The size of the sample population may vary and is based on the application. In some embodiments, biological sample collected from the population includes blood, saliva, or tissue samples. Proper handling of the sample ensures proper handling to preserve deoxyribonucleic acid (DNA) integrity.

In some embodiments, the deoxyribonucleic acid (DNA) may be extracted from the samples collected using standard protocols and kits suited for the sample type such as, blood DNA extraction kits and saliva deoxyribonucleic acid (DNA) extraction kits. In some embodiments,

In some embodiments, the genotyping may be performed on the deoxyribonucleic acid (DNA) extracted using genotyping arrays or platforms, such as microarrays to analyse genetic variants at specific loci or genome-wide. The sequencing may be performed on the deoxyribonucleic acid (DNA) extracted using techniques such as whole-genome sequencing (WGS), whole-exome sequencing (WES), or targeted sequencing such as, amplicon sequencing, targeted capture sequencing.

At 104, storing the genetic data obtained from a population.

At 106, processing the genetic data to identify genetic variants.

The processing of the stored genetic data includes aligning the genetic data to a reference genome to identify sequence variations and annotating the identified variants with genomic features such as gene annotations, functional domains, and regulatory elements.

At 108, identifying genetic variants, including single nucleotide polymorphisms (SNPs), insertions, deletions, and structural variations.

In some embodiments, identifying genetic variants includes mapping reads to genomic coordinates to identify potential genetic variants like, single nucleotide polymorphisms (SNPs), insertions, deletions (indels), and structural variations.

At 110, using statistical analysis to identify genetic variants that are associated with the trait or disease of interest.

At 112, visualizing the results through an interactive graphical interface 214.

The visualization of the results includes generating visual representations of variant data, including frequency plots, genotype-phenotype correlations, and pathway enrichment maps.

In some embodiments, the method 100 may include pre-processing the stored data prior to the processing of the data. The data preprocessing may include performing quality control checks to filter out low-quality reads, adapter sequences, and sequencing artifacts, filter reads based on quality scores, read length, or other pre-defined criteria, align reads to a reference genome or transcriptome using alignment algorithms such as Bowtie, Burrows-Wheeler Aligner (BWA), Spliced Transcripts Alignment to a Reference (STAR), and so, on.

FIG. 2 is a block diagram illustrating a computer-implemented system 200 for identifying genetic variants associated with a complex trait or disease, in accordance with an embodiment of the present invention.

The system 200 comprising a data collection module 202, a data storage module 204, a processing assembly 206, a variant identification module 208, a statistical analysis module 210, and a visualization module 212, a graphical interface 214, and a communication network 216.

The data collection module 202 may collect genetic data including genomic data, transcriptomic data, epigenomic data, proteomic data, and/or any combination thereof.

In some embodiments, the data collection module 202 may include sample collection, deoxyribonucleic acid (DNA) extraction, genotyping or sequencing of the extracted deoxyribonucleic acid (DNA).

The data storage module 204 may be linked to the data collection module 202 and the data storage module 204 configured to store genetic data obtained from a population.

The data storage module 204 for the genetic data may be configured as a scalable and reliable storage solution capable of handling large volumes of genetic data and efficient storage enabling retrieval of large volumes of genetic data while maintaining data integrity.

In a preferred embodiment, the data storage module 204 may support appropriate file formats for storing genetic data, such as FASTA (for nucleotide sequences), FASTQ (for sequencing reads), VCF (Variant Call Format), BAM (Binary Alignment/Map), or BED (Browser Extensible Data). In some embodiments, the data storage module 204 may ensure compatibility with bioinformatics tools and external databases used for analysis and visualization. The data storage module 204 may ensure interoperability and data harmonization across different data sources and formats.

In some embodiments, the data storage module 204 may be any cloud storage such as, AWS S3, Google Cloud Storage, Azure Blob Storage or on-location storage systems such as, network attached storage (NAS) and storage area network (SAN). In some embodiments, the data storage module 204 may implement security measures to protect genetic data from unauthorized access, data breaches, or cyberattacks by using encryption protocols such as SSL (secure sockets layer) and TLS (transport layer security). In a preferred embodiment, the data storage module 204 may store metadata along with genetic data, including sample information, experimental protocols, quality metrics, and analysis parameters.

The processing assembly 206 may be linked to the data storage module 204 and the processing assembly 206 may further comprise the variant identification module 208, the statistical analysis module 210, and the visualization module 212.

The variant identification module 208 may be linked to the data storage module 204 and the variant identification module 208 is configured to process the genetic data to identify genetic variants in the population.

The variant identification module 208 may use suitable technique to identify genetic variants, such as, read alignment, variant calling, and/or haplotype phasing.

The genetic data variants may include single nucleotide polymorphisms (SNPs), copy number variations (CNVs), insertions, deletions, and/or any other type of genetic variation.

The variant identification module 208 may use suitable technique to identify genetic variants, such as, read alignment, variant calling, and/or haplotype phasing.

In a preferred embodiment, the variant identification module 208 may accurately detect, annotate, prioritize, and visualize genetic variants within genomic data, leading to insights into genotype-phenotype associations, disease mechanisms, and therapeutic targets. In some embodiments, the variant identification module 208 may identify variants by comparing aligned reads to the reference genome. The variant identification module 208 may use variant calling algorithms such as, Genome Analysis Toolkit (GATK), FreeBayes, VarScan and/or others to detect genetic variants based on read alignments, coverage, base quality, and variant frequencies.

In some embodiments, the variant identification module 208 may perform joint variant calling for multiple samples or populations to improve variant detection sensitivity and accuracy. In some embodiments, the variant identification module 208 may annotate identified variants with genomic features such as gene names, functional consequences synonymous, or non-synonymous, impact on protein structure, and predicted pathogenicity. In an embodiment of the present disclosure, the variant identification module 208 may retrieve annotations from external databases such as, dbSNP, ClinVar, COSMIC.

In some embodiments, the variant identification module 208 may use quality filters to remove low-confidence variants, including filtering based on read depth, mapping quality, strand bias, and variant allele frequency. In some embodiments, the variant identification module 208 may perform recalibration and quality score adjustments to improve variant calling accuracy and reliability. In some embodiments, the variant identification module 208 may prioritize variants based on predicted functional impact, conservation across species, allele frequencies in population databases, and relevance to phenotype or disease.

The statistical analysis module 210 may be linked to the variant identification module 208 and the statistical analysis module 210 configured to analyze the genetic variants identified by the variant identification module 208 and determine which variants are associated with the trait or disease of interest.

The statistical analysis module 210 may use any suitable technique to perform statistical analysis, including logistic regression, linear regression, or Bayesian analysis.

The statistical analysis module 210 may be configured to correct for population stratification, perform genome-wide multiple testing correction, and incorporate functional annotation information.

In an embodiment of the present disclosure, the statistical analysis module 210 may cater to different aspects of genetic variant identification and analysis, including quality control, association testing, population genetics, and others. In some embodiments, the statistical analysis module 210 may perform various analyses such as case-control association, quantitative trait association, manipulating variant data, performing association tests, and such.

In an embodiment of the present disclosure, the statistical analysis module 210 may employ PLINK for handling large datasets and supports various file formats for genetic data, Genome Analysis Toolkit (GATK) for variant discovery and genotyping. It provides modules for variant calling, quality control, and for analyzing high-throughput sequencing data, such as whole-genome sequencing and exome sequencing, VariantTools.jl suitable for large-scale genomic analyses such as, manipulating variant data, performing association tests, and visualizing results, Penalized Logistic Regression for identifying genetic markers associated with disease or trait outcomes.

The visualization module 212 may be linked to the statistical analysis module 210 and the visualization module 212 configured to generate visualizations of the genetic variants identified by the variant identification module 210 and the statistical analysis results generated by the statistical analysis module.

The visualization module 212 may facilitate interpretation of the statistical analysis result and identification of potential functional mechanisms underlying the genetic variants.

In a preferred embodiment, the visualizations may include Manhattan plots, QQ plots, and forest plots, among others. In some embodiments, the visualization module 212 may generate visualizations for the results obtained from the variant identification module 208, such as, variant frequency plots, genotype-phenotype associations, and genome browser tracks. In some embodiments, the visualization module 212 may have integrated visualization tools such as, Integrative Genomics Viewer (IGV), and GenomeBrowse for interactive exploration of genomic regions, variant annotations, and sequence alignments. In some embodiments, the visualization module 212 may generate variant call files or variant annotation files containing detailed information about identified variants.

In some embodiments, the visualization module 212 may offer a visual depiction of genes expression facilitating identification of clusters of genes with similar expression patterns. The visualization module 212 may elucidate on complex patterns and relationships within the data. The visualization module 212 may employ a plurality of visualization technique, including Heatmaps coupled, Hierarchical Clustering, Volcano Plots, Scatter Plots, Chromatin Interaction Maps, Enrichment Plots, Pathway Maps and/or combination thereof.

In some embodiments, the visualization module 212 may depict interactions between genes, proteins, or other biological entities, providing insights into regulatory networks, protein-protein interactions, and pathways involved in disease mechanisms. In some embodiments, the visualization module 212 may enable the visualization of spatial interactions between chromatin regions and uncovering higher-order chromatin structures. In some embodiments, the visualization module 212 may be utilized to annotate genes, identify enriched biological pathways, and visualize pathway relationships. In some embodiments, the visualization module 212 may create Interactive Dashboards and allow users to interactively explore and analyze genetic variant data.

The graphical interface 214 may be linked to the visualization module 212 and the graphical interface 214 configured to visualizing the results obtained.

In some embodiments, the graphical interface 214 may be any a user device may be any desktop computer, laptop computer, a user computer, tablet computer, a personal digital assistant (PDA), any smart digital interactive screen cellular telephone, or a combination of any these data processing devices or any other data processing devices. The user device may comprise, a memory, a processor, and capability to connect with the communication network 214.

The communication network 216 may be linking the data collection module 202 to the data storage module 204 and the communication network 216 configured to enable the transfer of raw genetic data and facilitate integration with external computing resources.

In a preferred embodiment, the communication network 216 enables the transfer of raw genomic data comprises genome sequences or genetic data repositories to the data storage module 204 within the system 100. The communication network 216 facilitates high-speed data transfers over local networks or secure data transmission protocols over the internet.

In a preferred embodiment, the communication network 216 enables the linkage of the visualization module 212 to the graphical interface. In some embodiments, the communication network 216 facilitates inter-communication between various components of the system 100 and seamless data flow and information exchange between modules during the processing.

In some embodiments, the external computing resources comprises cloud computing platforms, bioinformatics tools, APIs (Application Programming Interfaces), third-party applications, reference genomes, annotation databases, clinical databases, and public repositories. The integration with external computing resources enhances the comprehensiveness and accuracy of genetic variant identification by leveraging diverse data sets. In a preferred embodiment, the communication network 216 allows remote access, data sharing and real-time reporting functionalities for analysis results, visualizations, and reports generated.

In some embodiments, the communication network 216 may include the Internet, a WI-FI connection, a wireless communication network, a 3G communication network, a 4G communication network, a 5G communication network, or any combination thereof any transceiver, or any combination thereof by triangulation, by a local positioning (LPS) device, by a global positioning system (GPS), or by any combination thereof. Embodiments of the present disclosure are intended to cover any remote communication technology, including known, prior art or later developed technologies.

A key advantage of the disclosed method 100 and system 200 is to enhance effectiveness and reliability of genetic variant identification. The proposed method 100 has more accuracy, sensitivity, specificity, and reproducibility compared to the traditional methods of gene variant detection.

A key advantage of the system 200 is compatible with standard bioinformatics tools, databases, and formats for seamless integration into existing workflows and analysis pipelines. Yet another advantage of the proposed system 200 is scalability to process data from diverse populations, cohorts, or studies with varying sample sizes and genetic complexity. Another advantage of the proposed system 200 is its robustness to variations in sequencing or genotyping platforms, data quality, and experimental conditions that can produce consistent results across different datasets and experimental setups.

The proposed system 200 and method 100 may provide information about variant type, genomic location, functional impact, allele frequencies, and potential disease associations. The proposed system 200 and method 100 may has quality control measure to filter out low-quality variants, and ensure the reliability of identified genetic variants. The proposed system 200 and method 100 may handle genetic data from diverse populations, account for population-specific variants, and avoid biases related to population structure.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

The foregoing descriptions of specific embodiments of the present technology have been presented for purposes of illustration and description. They are not intended to be exhaustive or to limit the present technology to the precise forms disclosed, and obviously many modifications and variations are possible in light of the above teaching. The embodiments were chosen and described in order to best explain the principles of the present technology and its practical application, to thereby enable others skilled in the art to best utilize the present technology and various embodiments with various modifications as are suited to the particular use contemplated. It is understood that various omissions and substitutions of equivalents are contemplated as circumstance may suggest or render expedient, but such are intended to cover the application or implementation without departing from the spirit or scope of the claims of the present technology.

What is claimed for:

1. A computer-implemented method for identifying genetic variants associated with a complex trait or disease, the method comprising:
obtaining genetic data from a population;
storing the genetic data obtained from the population;
processing the genetic data to identify genetic variants;
identifying genetic variants, including single nucleotide polymorphisms (SNPs), insertions, deletions, and structural variations;
using statistical analysis to identify genetic variants that are associated with the trait or disease of interest; and
visualizing the results through an interactive graphical interface.

2. The method of claim 1, wherein the genetic data is obtained using a technique comprises next-generation sequencing, microarray analysis, polymerase chain reaction (PCR), or any combination thereof.

3. The method of claim 1, wherein the processing of the stored genetic data comprises:
aligning the genetic data to a reference genome to identify sequence variations; and
annotating the identified variants with genomic features such as gene annotations, functional domains, and regulatory elements.

4. The method of claim 1, wherein the visualization of the results includes generating visual representations of variant data, including frequency plots, genotype-phenotype correlations, and pathway enrichment maps.

5. A computer-implemented system for identifying genetic variants associated with a complex trait or disease, the system comprising:
a data collection module;
a data storage module linked to the data collection module, the data storage module configured to store genetic data obtained from a population;
a processing assembly linked to the data storage module, the processing assembly further comprises:
a variant identification module linked to the data storage module, the variant identification module is configured to process the genetic data to identify genetic variants in the population;
a statistical analysis module linked to the variant identification module, the statistical analysis module configured to analyze the genetic variants identified by the variant identification module and determine which variants are associated with the trait or disease of interest; and
a visualization module linked to the statistical analysis module, the visualization module configured to generate visualizations of the genetic variants identified by the variant identification module and the statistical analysis results generated by the statistical analysis module;
a graphical interface linked to the visualization module, the graphical interface configured to visualizing the results obtained.

6. The system of claim 5, wherein the system further comprises a communication network linking the data collection module to the data storage module, the communication network configured to:
enable the transfer of raw genetic data; and
facilitate integration with external computing resources.

7. The system of claim 5, wherein the data collection module collects genetic data including genomic data, transcriptomic data, epigenomic data, proteomic data, and/or any combination thereof.

8. The system of claim 5, wherein the genetic data variants comprises single nucleotide polymorphisms (SNPs), copy number variations (CNVs), insertions, deletions, or any other type of genetic variation.

9. The system of claim 5, wherein the data storage module for the genetic data is configured to be:
scalable and reliable storage solution capable of handling large volumes of genetic data; and
efficient storage enabling retrieval of large volumes of genetic data while maintaining data integrity.

10. The system of claim 5, wherein the variant identification module uses suitable technique to identify genetic variants, such as, read alignment, variant calling, and/or haplotype phasing.

11. The system of claim 5, wherein the statistical analysis module uses any suitable technique to perform statistical analysis, including logistic regression, linear regression, and/or bayesian analysis.

12. The system of claim 5, wherein the statistical analysis module is configured to correct for population stratification.

13. The system of claim 5, wherein the statistical analysis module is configured to perform genome-wide multiple testing correction.

14. The system of claim 5, wherein the statistical analysis module is configured to incorporate functional annotation information.

15. The system of claim 5, wherein the visualization module facilitates interpretation of the statistical analysis result and identification of potential functional mechanisms underlying the genetic variants.
